(19) United States
(12) Patent Application Publication
(10) Pub. No.: US 2010/0323033 A1
(43) Pub. Date: Dec. 23, 2010

(54) CANCER SENSITIZER COMPRISING CHLOROGENIC ACID

(76) Inventors: Soo Youl Kim, Seoul (KR); Kang-Seo Park, Gyeonggi-do (KR); Kyung chae Jeong, Gyeonggi-do (KR)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 12/740,978

(22) PCT Filed: Oct. 1, 2008
(86) PCT No.: PCT/KR2008/005798

§ 371 Date: Aug. 20, 2010

(30) Foreign Application Priority Data
Oct. 1, 2007 (KR) 10-2007-0098862

(51) Int. Cl. A61K 33/24; C07C 69/732; A61K 31/225; A61K 31/675; A61K 31/282; A61K 31/704; A61K 31/351; A61K 38/14; A61K 31/437; A61K 31/475; A61K 31/337; A61K 31/7048; A61K 31/7028; A61K 31/255; A61K 31/407; A61K 31/381; A61P 35/00

(52) U.S. Cl. 424/649; 560/61; 514/533; 514/90; 514/492; 514/34; 514/459; 514/19.3; 514/285; 514/449; 514/27; 514/283; 514/42; 514/517; 514/83; 514/410; 514/432

(57) ABSTRACT

The present invention relates to a cancer sensitizer comprising chlorogenic acid or a derivative thereof. In particular, the present invention relates to a cancer sensitizer comprising chlorogenic acid or a derivative thereof, which can make cancer cells sensitive to anticancer agents to increase the therapeutic effect of anticancer agents. In addition, the present invention relates to a composition for inhibiting the chemo-resistance of cancer cells, comprising chlorogenic acid or a derivative thereof in combination with a pharmaceutically acceptable carrier, an anticancer composition comprising an anticancer agent in addition to the composition, and a method for disrupting the chemo-resistance of cancer cells, comprising administration of the cancer sensitizer.

### TECHNICAL FIELD

The present invention relates to a cancer sensitizer comprising chlorogenic acid or a derivative thereof. In particular, the present invention relates to a cancer sensitizer comprising chlorogenic acid or a derivative thereof, which can make cancer cells sensitive to anticancer agents to increase the therapeutic effect of said agents. In addition, the present invention relates to a composition for inhibiting the chemo-resistance of cancer cells, comprising chlorogenic acid or a derivative thereof in combination with a pharmaceutically acceptable carrier, an anticancer composition comprising an anticancer agent in addition to the composition, and a method for disrupting the chemo-resistance of cancer cells, comprising administration of the cancer sensitizer.

### BACKGROUND ART

Despite the large number thereof, anticancer agents developed thus far can treat only a few cancers completely. The reason why most cancers cannot be treated completely is that cancer is resistant to anticancer agents, or that tumors are reduced in size in the early stages of chemotherapy, but becomes resistant to anticancer agents during or after chemotherapy. In order to effectively treat cancer with anticancer agents, chemo-resistance, that is, the resistance of cancer cells to chemicals, must be overcome.

On the other hand, chlorogenic acid is an ester of caffeic acid and quinic acid, a major phenolic compound in coffee, and isolated from the leaves and fruits of dicotyledonous plants. Especially, chlorogenic acid is present in the green or roasted coffee bean, and can be readily extracted using a mixed solution of methanol and water by HPLC-UV [Bicchi et al., J. Agric. Food Chem., 43, pp 1549-1555 (1995)]. In addition, its isolation method and genealogical classification are disclosed by Clifford et al. in Phytochemistry, Vol. 28, No. 3, pp 829-838 (1989). This compound is an important factor in plant metabolism, known as an antioxidant, and also known to slow the release of glucose into the bloodstream after a meal.

In addition, a natural or synthesized chlorogenic acid is used for preventing or treating the decline of male reproductive function against hormone-disrupting chemicals (Korean Patent Publication No. 10-2001-87593), and chlorogenic acid extracted from coffee, nandina leaf, or unripe apple fruit is also used for preventing and treating hypertension (Japanese Patent Publication No. 2002-363075). Further, disclosed is a coffee drink composition, containing chlorogenic acid, and in which the content of hydroxyhydroquinone is reduced, having an excellent effect of reducing blood pressure (Korean Patent Publication No. 10-2006-128907). However, there is no mention of its activity as a cancer sensitizer in the prior arts.

### DISCLOSURE

### Technical Problem

Leading to the present invention, intensive and thorough research into effective chemotherapy, conducted by the present inventors, resulted in the finding that chlorogenic acid or derivatives thereof can reduce the chemo-resistance of cancer cells.

### Technical Solution

It is an object of the present invention to provide a cancer sensitizer comprising chlorogenic acid or a derivative thereof.

It is another object of the present invention to provide a composition for inhibiting the chemo-resistance of cancer cells, comprising chlorogenic acid or a derivative thereof, in combination with a pharmaceutically acceptable carrier.

It is still another object of the present invention to provide an anticancer composition, capable of inhibiting the chemo-resistance of cancer cells, comprising an anticancer agent in addition to the composition for inhibiting the chemo-resistance of cancer cells.

It is still another object of the present invention to provide a method for disrupting the chemo-resistance of cancer cells, comprising administration of the cancer sensitizer comprising chlorogenic acid or a derivative thereof, or the composition for inhibiting the chemo-resistance of cancer cells.

### DESCRIPTION OF DRAWINGS

FIG. 1 is the result of XTT assay to examine the effect of chlorogenic acid on sensitivity to an anticancer agent in human pancreatic carcinoma cell line AsPC-1.

### BEST MODE

In accordance with an aspect, the present invention pertains to a cancer sensitizer comprising chlorogenic acid or a derivative thereof.

As used herein, the term “cancer sensitization” is intended to have the same meaning as “chemical sensitization”, including the effect of, upon chemotherapy, improving or increasing the cytotoxicity of the anticancer agents on cancer cells in the presence of, rather than the absence of, a cancer sensitizer, that is, the effect of decreasing the resistance of cancer cells to chemicals, as in the case of chemo-resistance, with the aid of a cancer sensitizer to increase the therapeutic effect of the anticancer agents. In the present invention, chlorogenic acid functions to make cancer cells sensitive to anticancer agents and decrease the resistance of cancer cells to anticancer agents, thereby increasing the therapeutic effect of anticancer agents. Therefore, the term “cancer sensitization” is used herein to have the meaning equivalent to “chemical sensitization.”

By the term “chemo-resistance”, as used herein, it is meant that anticancer drugs, when administered, cannot kill cancer cells at all, or can regulate cancer cells only to a slight extent mainly due to the resistance of cancer cells to chemical drugs. On the whole, the term “resistance to chemical drugs” is intended to mean that when cancer patients are treated with anticancer drugs, there are no therapeutic effects, or the therapeutic effect of the anticancer drugs, although high in the early stages of chemotherapy, is progressively attenuated with continual treatment. It is well known in the art that the therapeutic effect of anticancer drugs is gradually decreased with the increasing number of administrations thereof. Chemo-resistance is attributed to the appearance of drug-resistant cells.

As mentioned above, chlorogenic acid is generally present in the leaves or fruits of dicotyledonous plants, and has the chemical name 3-[[3-(3,4-dihydroxyphenyl)-1-oxo-2-propenyl]oxy]-1,4,5-trihydroxycyclohexanecarboxylic acid. It can be also named as 3-caffeoylquinic acid. A small amount of isomers, such as isochlorogenic acid and neochlorogenic acid, is contained in naturally present chlorogenic acid, and caffeic acid is produced at the time of hydrolysis. Chlorogenic acid has a molecular formula of C₁₆H₁₈O₉, a molecular weight of 354.30, and the following structural formula.

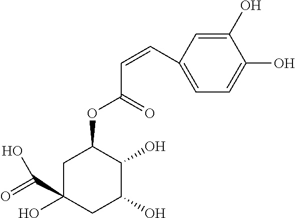

In the present invention, a natural or synthesized chlorogenic acid may be used, and the natural chlorogenic acid may be obtained by purifying or isolating the natural phenol and polyphenol extracted from Rosaceae fruits such as apple, pear and peach, coffee bean, cacao bean, seed of grape, and artichoke according to the known method (H. Li et al., J. Chromatogr. A 1098 (2005) 66-74 and V. Ossipov et al., J. Chromatogr. A 721 (1996) 59-68 etc.). In addition, the synthesized chlorogenic acid may be obtained synthetically according to a known method (M. Lepelley et al., Plant Science 172 (2007) 978-996 and J. Stockigt et al., FEBS LETTERS 42 (1974) 131-134). The natural or synthesized chlorogenic acid may be directly prepared or commercially available.

The chlorogenic acid derivative is an ester form isolated from a natural source, known as methyl chlorogenate and ethyl chlorogenate, and U.S. Pat. No. 6,632,459 discloses a chlorogenic acid derivative, in which a hydroxyl group of chlorogenic acid is substituted with caffeic acid. The cancer sensitizer according to the present invention includes chlorogenic acid and a derivative thereof which has the effect of cancer sensitizer being equivalent to chlorogenic acid.

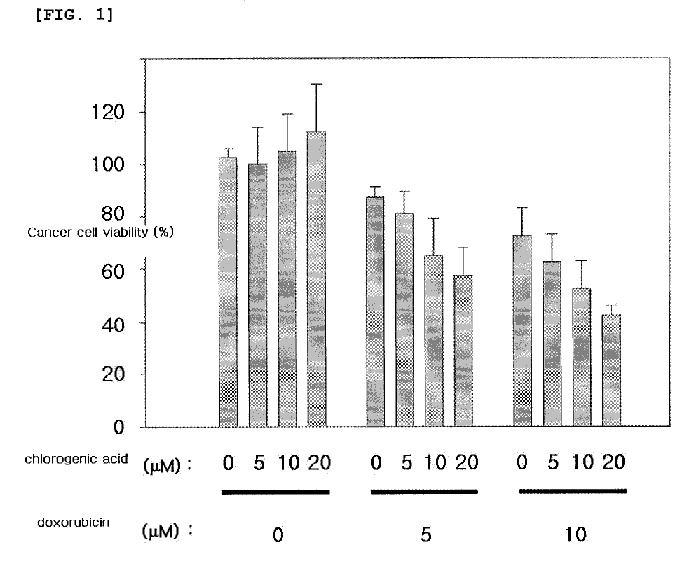

It is found that when an anticancer agent is administered in combination with chlorogenic acid, the viability of cancer cells showing chemo-resistance is 30% lower than that of cancer cells when it is administered alone, as shown in FIG. 1. These results demonstrate that chlorogenic acid has an effect of cancer sensitization.

No particular limitations are imposed on the kind of cancers for which chlorogenic acid or derivatives thereof can be used as a cancer sensitizer. Therefore, the kind of cancer to which the composition comprising an anticancer drug and chlorogenic acid in accordance with the present invention can be applied is dependent on the anticancer drug. For example, chlorogenic acid may be used as a cancer sensitizer in combination with cisplatin for the treatment of testicular cancer, ovarian cancer, lung cancer, head and neck cancer, cervical spinal cord tumor, neuroblastoma, osteosarcoma, etc., in combination with doxorubicin for the treatment of breast cancer, endometrial cancer, head and neck cancer, Ewing's sarcoma, osteosarcoma, leukemia, etc., in combination with etoposide for the treatment of lung cancer, testicular cancer, osteosarcoma, leukemia, neuroblastoma, etc.

In accordance with another aspect, the present invention pertains to a composition for inhibiting chemo-resistance (enhancing chemical sensitization), comprising chlorogenic acid or a derivative thereof, and a pharmaceutically acceptable carrier.

As used herein, the term “pharmaceutically acceptable carrier” refers to a carrier or diluent that does not cause significant irritation to a subject and does not abrogate the biological activity and properties of the administered compound. This composition may be formulated, together with a carrier, into dosage forms. Examples of oral dosage forms include troches, lozenges, aqueous or emulsified suspensions, powder, granules, emulsions, hard or soft capsules, syrups, and elixirs, but are not limited thereto. Useful for the preparation of tablets or capsules are a binder such as lactose, saccharose, sorbitol, mannitol, starch, amylopectin, cellulose and gelatin, an expedient such as dicalcium phosphate, a disintegrant such as corn starch and sweet potato starch, and a lubricant such as magnesium stearate, calcium stearate, sodium stearyl fumarate and polyethylene glycol wax. For capsules, a liquid carrier such as a lipid may be further used in addition to the above-mentioned compounds.

For non-oral administration, chlorogenic acid may be formulated into injections for subcutaneous, intravenous or intramuscular routes, suppositories, or sprays inhalable via the respiratory tract, such as aerosols. Injection preparations may be obtained by dissolving or suspending chlorogenic acid, together with a stabilizer or a buffer, in water and packaging the solution or suspension in ampules or vial units. Suppositories are typically made of a suppository base, such as cocoa butter and another glyceride, or a therapeutic laxative. For sprays such as aerosol, a propellant for spraying a water-dispersed concentrate or wetting powder may be used in combination with an additive.

In accordance with still another aspect, the present invention pertains to an anticancer composition, capable of inhibiting the chemo-resistance of cancer cells, comprising an anticancer agent in addition to the composition for inhibiting chemo-resistance.

The term “anticancer agent”, as used herein, is intended to refer to a chemical that can kill cancer cells. Most anticancer agents play a critical role in blocking the replication, transcription or translation of DNA in cancer cells. The kind of anticancer agent that can be used in the composition of the present invention is not particularly limited. Anticancer agents may be selected under standard considerations, such as the kind of cancer cells, the absorption rate of the drug (treatment time period and administration route), the position and size of tumors, etc. For instance, anticancer agents useful in the present invention may be DNA alkylating agents such as mechlorethamine, chlorambucil, phenylalanine, mustard, cyclophosphamide, ifosfamide, carmustine (BCNU), lomustine (CCNU), streptozotocin, busulfan, thiotepa, cisplatin, and carboplatin. Anti-cancer antibiotics are also used in the present invention, exemplified by dactinomycin (actinomycin D), doxorubicin (adriamycin), daunorubicin, idarubicin, mitoxantrone, plicamycin, mitomycin and C Bleomycin. Examples of plant alkaloids as anticancer agents useful in the present invention include vincristine, vinblastine, paclitaxel, docetaxel, etoposide, teniposide, topotecan and irinotecan. However, anticancer agents useful in the present invention are not limited to the aforementioned compounds.

In accordance with still another aspect, the present invention pertains to a method for disrupting the chemo-resistance of cancer cells, comprising administration of the cancer sensitizer comprising chlorogenic acid, or the composition for inhibiting the chemo-resistance of cancer cells.

The term “administration,” as used herein, is intended to refer to the introduction of the cancer sensitizer of the present invention into cancer patients in a suitable manner. As long as it leads the cancer sensitizer to a desired tissue, any administration route may be adopted. For example, the administration of the cancer sensitizer may use oral, intraperitoneal, intravenous, intramuscular, intracutaneous, subcutaneous, intranasal, intrapulmonary, intrarectal, intrathecal, or intradural routes, but is not limited thereto. The cancer sensitizer according to the present invention may be administered simultaneously with or separately from an anticancer agent. In the latter case, the cancer sensitizer may be administered at predetermined time intervals before or after the administration of an anticancer agent. Preferably, the cancer sensitizer may be administered after anticancer agents. The cancer sensitizer of the present invention may be administered once a day, or two or three times a day at predetermined intervals.

In consideration of various factors including the kind of cancer, administration routes, therapeutic effects and chemical sensitization, the cancer sensitizer or the anticancer composition according to the present invention may be suitably administered.

### MODE FOR INVENTION

A better understanding of the present invention may be obtained through the following examples, which are set forth to illustrate, but are not to be construed as the limit of the present invention.

### Example 1

### Preparation and Treatment of Cells Resistant to Anticancer Agent

Human pancreatic carcinoma cell line AsPC-1 (doxorubicin resistant) was purchased from ATCC (American Type Culture Collection, Manassas, Va., USA). AsPC-1 cell lines were maintained in media supplemented with 10% fetal bovine serum (Gibco BRL) at 37° C. in a humid 5% CO₂ atmosphere, and subcultured every two or three days.

### Example 2

### Effect of Chlorogenic Acid on Anticancer Agent Sensitivity

XTT assay was conducted to examine whether chlorogenic acid increases the anticancer agent sensitivity. A modified MTT assay, XTT assay is a colorimetric method that determines the relative number of living cells, after treating cells with a compound degraded by a specific enzyme.

AsPC-1 cells were seeded in 96-well plates (2×10⁴ per well), and cultured for 24 hrs. The cells were treated with 0, 5, and 10 μM of doxorubicin, and 0, 5, 10, and 20μ of chlorogenic acid, and incubated for 48 hrs. 0.1 ml of active solution and 5 ml of XTT (2,3-Bis{2-methoxy-4-nitro-5[(sulfenylamino) carbonyl]-2H-tetrazolium-hydroxide}) were added to the 96-well plates, and 50 μl of reaction solution was added to each well, followed by incubation for 2 hrs. The plates were gently shaken for staining, and the absorbance was measured at 450 nm using a VERSAmax220 Automatic Microplate Reader (Molecular Devices Corp.). Viability of cancer cells is shown in FIG. 1.

As shown in FIG. 1, the viability of cancer cells administered both chlorogenic acid and the anticancer agent was decreased by 30% more, as compared to that of cancer cells when the anticancer agent was administered alone. The viability of cancer cell was decreased, as the concentration of chlorogenic acid was increased. These results demonstrate that chlorogenic acid has an effect of cancer sensitization.

### INDUSTRIAL APPLICABILITY

A naturally occurring, harmless material, chlorogenic acid or its derivatives can be used as a cancer sensitizer which makes cancer cells highly sensitive to anticancer agents without the production of particular side effects, in contrast to conventional chemical sensitizers.

1. A cancer sensitizer comprising chlorogenic acid or a derivative thereof.

2. A composition for inhibiting the chemo-resistance of cancer cells, comprising chlorogenic acid or a derivative thereof in combination with a pharmaceutically acceptable carrier.

3. The anticancer composition comprising the composition of claim 2 and an additional anticancer agent.

4. The anticancer composition according to claim 3, wherein the anticancer agent is selected from the group consisting of mechloethamine, chlorambucil, phenylalanine, mustard, cyclophosphamide, Ifosfamide, carmustine (BCNU), lomustine (CCNU), streptozotocin, busulfan, thiotepa, cisplatin, carboplatin, dactinomycin (actinomycin D), doxorubicin (adriamycin), daunorubicin, idarubicin, mitoxantrone, plicamycin, mitomycin, C Bleomycin, vincristine, vinblastine, paclitaxel, docetaxel, etoposide, teniposide, topotecan, and iridotecan.

5. The method for disrupting the chemo-resistance of cancer cells, comprising administering the cancer sensitizer of claim 1.

6. The method for disrupting the chemo-resistance of cancer cells, comprising administering the composition for inhibiting the chemo-resistance of cancer cells of claim 2.
